(19) United States
(12) Patent Application Publication
(10) Pub. No.: US 2025/0177299 A1
(43) Pub. Date: Jun. 5, 2025

(54) MOLECULAR COMPLEXING METHOD, FORMULATION AND MANUFACTURING FOR ENHANCED NUTRIENT DELIVERY

(71) Applicant: Ingredient Fusion, LLC, Phoenix, AZ (US)

(72) Inventor: Stephen LaRue DURKEE, Maricopa, AZ (US)

(73) Assignee: Ingredient Fusion, LLC, Phoenix, AZ (US)

(21) Appl. No.: 18/724,981

(22) PCT Filed: Dec. 19, 2022
(86) PCT No.: PCT/US2022/053343

§ 371 Date: Jun. 27, 2024

Related U.S. Application Data
(60) Provisional application No. 63/295,629, filed on Dec. 31, 2021.

(51) Int. Cl. A61K 9/107; A61K 31/353; A61K 31/593; A61K 31/7028; A61K 36/67; A61K 36/9066; A61K 47/14; A61K 47/26; A61K 47/36

(52) U.S. Cl. CPC A61K 9/1075; A61K 31/353; A61K 31/593; A61K 31/7028; A61K 36/67; A61K 36/9066; A61K 47/14; A61K 47/26; A61K 47/36

(57) ABSTRACT

A method of preparing an emulsion of a bioactive agent having enhanced bioavailability, the method comprising admixing a particulate bioactive agent with a lipid to form a mixture, adding water to the mixture to form a suspension of the bioactive agent and the lipid, reducing a particle size of the bioactive agent in the suspension to 1.0 μm or less, and adding a surface active agent to the resulting suspension of the bioactive agent to create the emulsion containing the bioactive agent having a particle size of 1.0 μm or less.

### CROSS-REFERENCE TO RELATED APPLICATION

This application claims priority under 35 U.S.C. 119 (e) to U.S. provisional application Ser. No. 63/295,629, filed on Dec. 31, 2021, which is incorporated herein by reference in its entirety.

### FIELD OF THE INVENTION

The present invention relates to an improved method of preparing a hydrophobic molecule in its base form to provide enhancements in bioavailability, emulsification, and suspension of natural substances. This improved method is primarily used for increasing the functional benefits of food and beverage, cosmetics, and other health products. The present invention also generally relates to processes and formulations for producing water soluble nano and micro-emulsions of targeted aglycones and other hydrophobic molecules. Finally, this invention relates to method, protocols, and formulations for preparation of fine and stable nano and micro-emulsions and suspensions, and improved products created therefrom.

### BACKGROUND OF THE INVENTION

The functionality of bioactive compounds present in foods, dietary supplements, or drugs largely depends on their fate in the gastrointestinal tract (GIT). The bioavailability of many health-promoting compounds is generally low due to their low bio accessibility, susceptibility to degradation, or poor absorption profile. Specifically, many hydrophobic bioactive compounds found in natural food products suffer from similar shortcomings as above—relatively low oral bioavailability due to their low bio accessibility, chemical instability, or poor absorption.

There are several strategies that can be used to enhance the liberation, solubilization and absorption of bioactive compounds using lipids. Colloidal delivery systems, for example, have been theorized and many methods have been proposed and tested with varying degrees of success. Other common examples of nano and micro-preparations used in the industry include SLN, LNC, Micelles, Lipospheres, Liposome and even more complex methods such as Ethosome, Niosome, and Transferosome. However, all these preparation methods suffer similar difficulties. These difficulties include the complexity of formulation, the heavy reliance on synthetic ingredients, extensive preparatory protocols, and outcomes can vary widely in many cases, leading to products not yet ready for industry or products that can be produced but only on a limited or customized basis. Thus, there is a need for the practical and improved creation and manufacturing of stable nano and micro-emulsions of high utility.

### SUMMARY OF THE INVENTION

An aspect of the present invention relates to an improved method of preparing a hydrophobic molecule in its base form to provide enhancements to form and function. Specifically, enhancements in form provides a solubilized bioactive being a compound that contains the bioactive, one or more dispersible lipid(s) (with additional functional properties as described below under functional enhancements) and one or more surface active substances (with additional functional properties also described under functional enhancements) that have affinity for both oil and water.

Enhancements in function provide a compound superior to its base form so that the compound's lipid(s) make the compound more easily dispersible in water, more readily used within an extracellular fluid, and more easily metabolized in a second path metabolism on a cellular level. Additionally, functional enhancement is provided by the addition of surface active substances that have affinity for water and oil so that an increased amount of lipid (loading rate of the substance) can be increased.

Accordingly, an enhanced dose of the bioactive can be delivered in an improved manner into the GIT compared to the bioactive in its base form. Thus, this method allows improvements in both form and function of water repelling substances (hydrophobic) of natural origin in aspects of bioavailability, bioactivity, emulsification and suspension, as well for commercial and industrial use.

In view of the above considerations, a more sustainable and cost-effect approach to enhancing the functionality of bioactive compounds is to leave them within their natural environment, in a way that enhances their bioavailability. An aspect of the present invention is using a modified excipient nano and micro-emulsion preparation process and method for increasing the bioavailability of bioactive components free of traditional synthetic chemical excipients. Nano and micro-emulsions present several advantages over other methods for this application, such as the ability to incorporate hydrophilic, amphiphilic, and lipophilic excipient ingredients, high physical stability, and rapid gastrointestinal digestibility.

The processes that govern oral bioavailability of bioactive compounds are complex and include drug, supplement, or food, solubilization within gastrointestinal fluids, transportation into or out of epithelial cells and/or, biochemical or chemical transformations. In another aspect of the present invention, this method enhances the bioavailability of lipophilic bioactive compounds found in nature. However, the same principles can also be applied to supplements and pharmaceuticals of which a primary bioactive is additionally hydrophobic and lipid soluble.

Examples of current pharmaceuticals that meet this criterion are found largely in the Biopharmaceutics classification system (BCS) under class IV compounds but can also be found in other classifications. This problem has grown over time in pharmaceutical sciences such that now approximately only 8% of new drugs being produced do not have issues with efficacy. The advantages disclosed herein can also be applied in traditional drug development as well. BCS Class IV compounds exhibit the least oral bioavailability, low solubility and low intestinal permeability among all pharmaceutical classes of drugs. Thus, for these drugs to be effective, there is a need for a more compatible and efficient delivery system which can further improve clinical outcomes by reducing toxicity and side effects from a lower dosage requirement.

An aspect of the invention disclosed herein includes the capability of expanding the use and enhancing the safety (through enhanced efficacy of smaller amounts) of already developed drug compounds. An exemplary benefit to current BCS class IV compounds would be paclitaxel. Paclitaxel is a chemotherapy drug first isolated in 1971 from the bark of the Pacific yew tree. Paclitaxel contains endophytic fungi that synthesize the paclitaxel, enhance the efficacy of the paclitaxel and lowers the dosage that could have profound benefits from approved uses in non-small cell lung cancer, ovarian cancer and breast cancer. Additional examples of already in use lipid soluble drugs that could be enhanced by the method disclosed herein include amphotericin B, furosemide, acetazolamide, and ritonavir. As described previously and described below in detail, the drug formulations of base bioactive molecules would additionally be improved from the further enhancements to both form and function.

Further, it is an aspect of the present invention to provide a highly efficient method of preparation of a lipid soluble molecule in its base form that provides an enhancement to bioavailability from substances found only in nature. These substances are unique in their structural characteristics to allow for simplification based on the method steps disclosed herein. On the other hand, industry typically uses fatty acids and esters combined with synthetic excipients. This method disclosed herein advantageously uses ingredients that function as supplemental nutrients, satisfy consumers' desires for a clean label (a label free of synthetics that includes a smaller amount of ingredients), and provide eco-conscious solutions for health and overall wellbeing.

It is also an aspect of the present invention to provide a method that significantly increases bioactivity by more effectively treating several diseased states including viral infection (such as influenzas, coronaviruses, and reduction of human papillomavirus), cancers and other neoplasms (such as melanoma, various carcinomas, and reduction of pancreatic adenocarcinoma). An important aspect to the enhancement of improved bioactivity includes an effective range in nanometers of particle size. The advantageously improved bioactivity requires a critical balance of factors to be maintained. These factors include the activity seeking to accomplish the treatment or reversal of the diseased condition or the homeostatic balancing of the system and the retention time in the body and the ability of the body to use the compound for its intended purpose. Additionally advantageous is that these parameters can be adjusted by altering a relative size distribution of the compound to be less than one-thousand-five-hundred nanometers (r<1500 nm).

The retention time in the body relates to a time post-absorption and an amount of time before a compound is expelled from excretion. Industry poorly measures retention time through bioavailibilty studies that measure an amount entering and exiting the blood stream. As a result, this area of science is vastly underexplored. This area of science is also challenging because fat soluble compounds, for example, can be transferred to the extracellular fluid, thus being contained in lymph fluid, interstitial fluid, or in blood plasma making fat soluble compounds more difficult to quantify as far as absorption.

One skilled in the art understands that the smaller the particle, the faster the onset of the nutrients in the body and elimination thereof. Thus, adjusting particle size is a novel aspect of the inventive method for retention of the bioactive agent in the body that is not practiced in the industry. Instead, industry and scientific research currently is focused on making the particle size as small as possible which can lead to less advantageous outcomes in disease treatment or health by limiting loading, limiting delivery amount and limiting rapid use and potential for rapid elimination or conversion of certain compounds.

While the benefits of particle size have been explored and other methods may have objectively achieved this particle size, the invention disclosed herein expands the art in multiple capacities allowing for new frontiers in industry and expanded exploration in the use of bioactive compounds found in nature. These bioactive compounds found in nature have been limited in exploration and limited in use for human applications because of their hydrophobicity.

Some exemplary benefits include improving control over the energetic embodiment of the liquid complex, controlling the outcome of a particle size consistently throughout a process, reducing total ingredients in the complex to reach stability, providing new balances of compounds within the mixture (thus increasing the loading and efficacy), and using common industrial methods through the use of the invention as a preparatory step. Alternately, both the pharmaceutical and nutraceutical industries have chosen to overcome the efficacy problem by simply increasing the dose delivered to very large quantities which can cause a wide array of side effects. For example, large quantities, even with non-toxic compounds, deliver compounds that result in experiences of constipation and distension.

The conventional wisdom of using a compound having a smaller particle size is not always correct because the smaller the particle, the greater the surface area. There is a direct correlation between particle size and how much of a compound can be retained in the suspension or emulsion. Further, the smaller the particle, the faster the peak onset but also the faster the elimination period (i.e. quickly used up). Accordingly, defining a specific particle size range is advantageous since the size range can additionally prevent agglomeration, separation or irreversible gelation caused by factors of both formulation and process. At the same time, the final application can be relied upon to dictate the effective particle size. Thus, the size range creates a wider variety of use and better economics achieved through other current methods.

In view of the above, an end result in each case or use condition has been studied. The end result analysis evaluates bioavailability of the compound and measures an effective treatment of the infection or virus, for example, via CT scans before and after treatment. The end result analysis aids in understanding the use and function of the compound at a cellular level, and the known benefits of each part or similar constituency found in other organisms. Accordingly, the method can be modified in each specific use for the health of natural organisms beyond human beings, as well as in early investigations in plant husbandry.

The foregoing and/or other aspects of the present invention can be achieved by providing a method of preparing an emulsion of a bioactive agent having enhanced bioavailability, the method comprising admixing a particulate bioactive agent with a lipid to form a mixture, adding water to the mixture to form a suspension of the bioactive agent and the lipid, reducing a particle size of the bioactive agent in the suspension to 1.0 μm or less, and adding a surface active agent to the resulting suspension of the bioactive agent to create the emulsion containing the bioactive agent having a particle size of 1.0 μm or less.

The foregoing and/or other aspects of the present invention can be further achieved by providing a method of preparing an emulsion of a bioactive agent having enhanced bioavailability, the method comprising preparing a particulate bioactive agent comprising quercetin aglycone and having a particle size of 210 μm or less, mixing the particulate bioactive agent with a lipid selected from the group consisting one of caproic acid, capric acid, caprylic acid, caproic triglycerides, capric triglycerides, caprylic triglycerides, and mixtures thereof to form a mixture at a ratio between 1:1 to 1:3 by weight (bioactive:lipid), saturating the bioactive agent in the lipid, adding water to the mixture to form a suspension of the lipid saturated bioactive agent, reducing a particle size of the particulate bioactive agent in the suspension to 1.0 μm or less, and adding a surface active agent to the resulting suspension to create an emulsion including the bioactive agent having a particle size of 1.0 μm or less, where the surface active agent is selected from the group consisting of inositol, short chain fructooligosaccharides, and mixtures thereof in ratio between 0.1 to 0.5 by weight of the lipid.

The foregoing and/or other aspects of the present invention can be also be achieved by providing a method of preparing an emulsion having enhanced bioavailability, the method comprising preparing a particulate bioactive agent comprising a particulate plant extract of green tea including at least 20% catechins by weight and having a particle size of 210 μm or less, mixing the particulate bioactive agent with a lipid selected from the group comprising one of caproic acid, capric acid, caprylic acid, caproic triglycerides, capric triglycerides, caprylic triglycerides, and mixtures thereof to form a mixture at a ratio between 1:2 to 1:3 by weight (bioactive:lipid), saturating the particulate mixture in the lipid, adding water to the mixture to form a suspension of the lipid saturated bioactive agent, reducing a particle size of the particulate bioactive agent in the suspension to 1.0 μm or less, and adding a surface active agent to the resulting suspension to create an emulsion including the bioactive agent having a particle size of 1.0 μm or less, where the surface active agent is selected from the group comprising one of inositol, short chain fructooligosaccharides, and mixtures thereof in a ratio between 0.1 to 0.5 by weight of the lipid, wherein the emulsion comprises the particulate plant extract having a portion of the particulate bioactive agent being hydrophobic.

In addition, the foregoing and/or other aspects of the present invention can be achieved by providing a method of preparing an emulsion having enhanced bioavailability, the method comprising preparing a bioactive agent including a first particulate plant extract of turmeric comprising at least 30% of curcumin by weight and having a particle size of 210 μm or less, and including a second particulate plant extract of black pepper comprising at least 80% piperine by weight and having a particle size of 210 μm or less, mixing the first and second particulate plant extracts to form a uniform dry mixture, mixing the dry mixture with a lipid selected from the group comprising one of caproic acid, capric acid, caprylic acid, caproic triglycerides, capric triglycerides, caprylic triglycerides, and mixtures thereof, saturating the particulate mixture in the lipid, adding water to the mixture to form a suspension of the lipid saturated bioactive agent, reducing a particle size of the particulate bioactive agent in the suspension to 1.0 μm or less, and adding a surface active agent to the resulting suspension to create an emulsion including the bioactive agent having a particle size of 1.0 μm or less, where the surface active agent is selected from the group comprising one of inositol, short chain fructooligosaccharides, and mixtures thereof, wherein the emulsion comprises the plant extract having a portion of the bioactive agent being hydrophobic.

In addition, the foregoing and/or other aspects of the present invention can also be achieved by providing a method of preparing an emulsion having enhanced bioavailability, the method comprising preparing a bioactive agent including a particulate plant extract of natto comprising at least 1% of menaquinone-7 by weight and having a particle size of 210 μm or less, measuring the percentage of lipid contained in the particulate plant extract, mixing the bioactive agent with a lipid selected from the group comprising one of caproic acid, capric acid, caprylic acid, caproic triglycerides, capric triglycerides, caprylic triglycerides, and mixtures thereof to form a mixture at a ratio between 2:1 to 1:1 by weight (bioactive:lipid), adding water to the mixture to form a suspension, reducing a particle size of the particulate bioactive agent in the suspension to 1.0 μm or less, and adding a surface active agent to the resulting suspension to create an emulsion including the bioactive agent having a particle size of 1.0 μm or less, where the surface active agent is selected from the group comprising one of inositol, short chain fructooligosaccharides, and mixtures thereof, wherein the emulsion comprises the plant extract having a portion of the bioactive agent being hydrophobic.

Additional and/or other aspects and advantages of the present invention will be set forth in the description that follows, or will be apparent from the description, or may be learned by practice of the invention.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates a flow diagram of the method steps used to prepare a bioavailable emulsion.

### DETAILED DESCRIPTION OF EXEMPLARY EMBODIMENTS

In the following description, various aspects of the present invention will be described. However, it will also be apparent to one skilled in the art that the present invention may be practiced without the specific details presented herein once the general understanding of the particle interaction is explored and learned as commonly understood. Furthermore, well known features may be omitted or simplified in order not to obscure the present invention.

Specifically, multiple formularies and processes have been discovered and disclosed in the last thirty years to enhance hydrophobic compounds that are lipophilic including the unique improvement disclosed herein. Despite a wide range of preparation methods (Publication A) and formulation combinations (Publication B), a consistent method of preparation at an industrial scale using natural bio-compostable materials is still identified as a current need of the industry (Publication C), including those that follow global regulatory standards for Good Manufacturing Practices (GMP) (Publication D). Currently, the wide variation between assimilation steps and formulary characteristics, as well as the consistently highlighted drawbacks make utility lacking in the art. Publications (A)-(D) identified above are cited below and are hereby incorporated by reference in their entirety.
Publication (A)—Kumar M, Bishnoi R S, Shukla A K, Jain C P. Techniques for Formulation of Nanoemulsion Drug Delivery System: A Review. Prev Nutr Food Sci. 2019; 24(3):225-234.
Publication (B)—Selvakumari Sreenathkumar. Current Updates On Global Phytoceuticals and Novel Phyto Drug Delivery System In Herbal Medicine [Online First], IntechOpen, May 28th 2021.
Publication (C)—Antony V. Samrot, Tan Chuan Scan, Teeshalini Kudaiyappan, Ummu Bisyarah, Anita Mirarmandi, Etel Faradjeva, Amira Abubakar, Hawwa Hashma Ali, J. Lavanya Agnes Angalene, S. Suresh Kumar, Production, characterization and application of nanocarriers made of polysaccharides, proteins, bio-polyesters and other biopolymers: A review, International Journal of Biological Macromolecules, Volume 165, Part B, 2020, Pages 3088-3105.
Publication (D)—Souto E B, Silva G F, Dias-Ferreira J, Zielinska A, Ventura F, Durazzo A, Lucarini M, Novellino E, Santini A. Nanopharmaceutics: Part II—Production Scales and Clinically Compliant Production Methods. Nanomaterials. 2020; 10(3):455.

However, the invention disclosed herein provides increased utility to accomplish industrial and product applications in a completely new paradigm of human activities that was previously not realized by one skilled in the art. The preparatory method and manufacturing process disclosed herein is advantageously simplified for universal application to hydrophobic compounds that are nutritive and not synthetic.

The primary obstacles commonly encountered in the industry include the preparation of the primary ingredients and the specific complexing method of doing so. This is not surprising because the vast array of compounds can lead to billions or more of possible combinations. Further, a myriad of issues can arise from the wrong combinations such as the difficulty of dispersion, gelation and rapid agglomeration. There are already existing methodologies understood by one skilled in the art that accomplish nano emulsions and suspensions using specific ingredients that differ from those proposed herein. However, these ingredients are typically synthetic, uniform, and easy to build a method of process around. Outside of these well-known methodologies, scientific exploration and research defines a wide array of alternative approaches which further complicates the industrial utility. This is because these well-known methodologies are often specific to one compound, have widely varying degrees of outcomes, and are often inconsistently performed at only an exploratory level. Thus, these well-known methodologies lack utility to industry as previously described herein.

Another significant factor that has prevented advancement in the industry includes the complexity of synthesis in the preparation method disclosed herein which require expert level domain experience across multiple disciplines. This factor can be challenging because those researching and proposing methods are typically experts in a particular disciplines (chemistry or advancing known processes and process parameters, for example). Hence, the researcher is not focused on the economics of the output. Instead, the researcher is focused on improving a result of a known compound or substituting a new chemical compound so it would improve a previous output. The preparation method disclosed herein advantageously applies a lean methodology that combines physics, chemistry and mechanical process and requires specific domain expertise in each discipline to create the invention disclosed herein.

Additionally, conventional wisdom on the stability of nano and micro-emulsions corresponds to Zeta potential, or the electro-kinetic potential in colloidal dispersions. In other words, stability correlates to an electrical potential of the colloidal dispersion (of the molecular complex). On the other hand, the preparation method disclosed herein advantageously shows that a one-year stability of a compound whereby the mean peak of the particle size is greater than 250 nm has a higher correlation to stability than Zeta potential.

In view of this understanding, many of the additional compounds to create a method of preparation were advantageously eliminated by theorizing a correlation between commonly used excipients and natural molecules having similar means of exchanging energy at a molecular level. This theory was understood after exploring a wide array of natural compounds that are commonly used in place of synthetics for stability and dispersion aids.

To expand current methods in the industry, a wide array of pectin in high methoxylated to low methoxylated, as well as blends and natural extracts were trialed and found to have high microbial activity over long term trials. Additionally, popular formularies like lecithin and glycerin were trialed. Lecithin was found to continue to expand in formulations eventually leading to separation. Glycerin was found to have very low stability unless used in very small quantities. Thus, Glycerin alone was not useful to the invention disclosed herein which requires higher loading rates to achieve improved efficacy and a simplified industrially scalable process.

Additionally, various oils were used containing fatty acids and/or fatty acid esters, and raw vegetable oils such as those understood by one of ordinary skill in the art. In response to various challenges, a deeper understanding of the formularies and methodologies led to the following contributions to the invention disclosed herein. Specific knowledge obtained includes an understanding of how to organize the use of the lipid, what is suitable for certain applications and added motivation to conduct an expanded search and trial of additional lipids to narrow down what is disclosed herein. Oils largely requiring complex and synthetic stability aids were eliminated for same reason aforementioned as they lacked an ability to achieve higher loading rates and greater stability through improved efficacy and a simplified industrially scalable process.

Also, the preparation method disclosed herein advantageously reduces the complexing method down to a few simple steps. Further details of the preparation method are described below in detail.

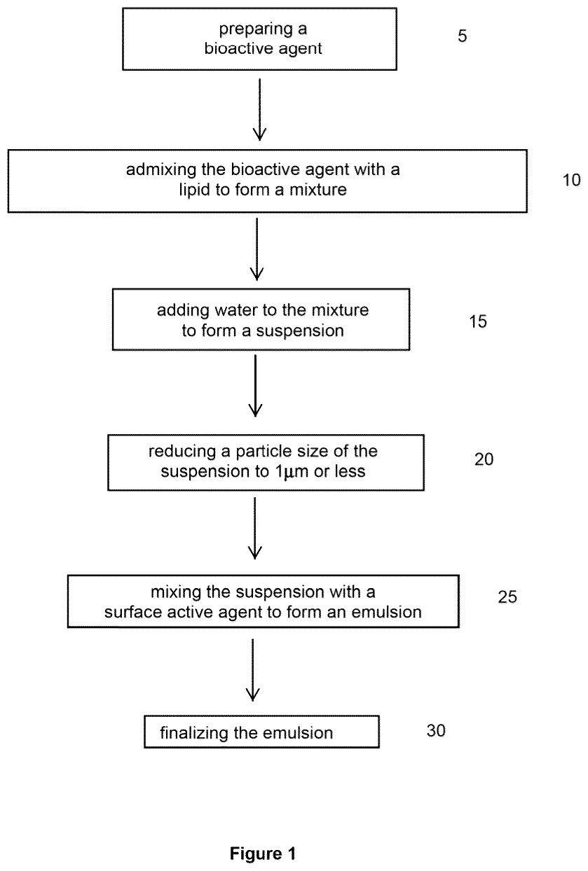

FIG. 1 illustrates the assembly/preparation method step of the enhanced bioavailable compound. The first step is to prepare a particulate bioactive agent including a primary bioactive (PB) or a primary bioactive extract (PBe) 5. The bioactive agent advantageously comprises nutritive ingredients that have an effect on a living organism, tissue or cell, including a natural compound derived from plants via extraction, expression or distillation without intentional chemical reactions or modification.

The bioactive agent is composed of a hydrophobic compound for biological use whereby the bioactive agent is lipophilic, the bioactive agent is at least partially solubilized in a lipid. Specifically, the bioactive agent includes a concentration of at least 20% by weight of a hydrophobic compound including hydrophobic constituents with a solubility less than 5 mg per ml in water. Such a configuration advantageously provides increased absorption in the body and higher efficacy compared to over 90% of industrial foods, health supplements, etc. as commonly understood by one skilled in the art.

A bioactive agent having less than 20% by weight of a hydrophobic compound has disadvantages for assimilating into the human body. Although human bodies rely on these types of bioactive agents for a wide range of metabolic activity, without lipid delivery systems, we cannot get enough into our systems. Specifically, challenges in food cultivation (nutrient loss from mass farming/distribution), food preparation (we rarely cook foods in a manner where the fat can assimilate with the nutrient) and food preservation (we rarely store foods through pickling and fermentation) reduce efficacy.

PB is preferably composed of a dried powder with a water content of less than 10% by weight. PBe includes multiple hydrophobic PBs and preferably established at a concentration of at least including 80% hydrophobic constituents by weight. Accordingly, PB is an active, natural and hydrophobic compound with limits on its bioactivity from lessened or poor bioavailability in an isolated or extracted form. However, by supplementing a delivery in a complex with other nutrients, the utility of the PB can be advantageously enhanced while lessening the amount needed to achieve the health benefit. Specifically, the complex is delivering other nutritive compounds along with the primary bioactive and the ones chosen here advantageously enhances the overall function of different systems. For example, a short chain fructooligosaccharides (sc-FOS) selectively targets key flora in the GIT to advantageously enhance longevity, stress tolerance, and immune function.

The bioactive agent is particulate bioactive agent and is preferably able to pass through a 100 mesh (0.0059 inch or 149 micron) screen in order to control a maximum particle size of 210 μm in the final product. Any bioactive agent that does not pass through the 100 mesh can preferably undergo additional grinding and/or sifting so that all of the bioactive agent can pass through the 100 mesh screen prior to use in the mixture. The bioactive agent sized to be less than 100 mesh and preferably less than 80 mesh advantageously creates stability.

A reduced particle size in a preparatory method is advantageously easier and faster to assimilate to the rest of the compound. Additionally, when high energy methods of shear are used, that energy transfers to a non-hydrophobic portion of the solution. Thus, preparing the hydrophobic portion in a size to be minimally sheared advantageously improves the stability of the final emulsion.

Moreover, energy balance advantageously improves loading. Specifically, the smaller the particle size, the less the non-hydrophobic solution can hold. As a result, there is an energetic balance translating to long term stability. There are specific particle size ranges that can advantageously achieve this balance as described herein, which are not understood in the industry.

Some exemplary hydrophobic compounds found in the particulate bioactive agent include Quercetin, Luteolin, Menaquinone-7, Piperine, Cannabinoids, Resveratrol and Zerumbone. Specifically, Quercetin includes one of a Quercetin Aglycone and Quercetin Dihydrate (CAS #6151-25-3). For example, Quercetin is highly hydrophobic with solubility of its aglycone (CAS #: 117-39-5) reported as less than 1 mg/mL at 70° F. In one embodiment, hydrophobic compounds in the bioactive agent include plant pigments such as anthocyanins, carotenoids, betalains, or flavonoids. The hydrophobic compounds in the bioactive agent can also include at least one fat soluble vitamin such as vitamin A, vitamin D, vitamin E and vitamin K. All of the compounds listed advantageously provide improved use and bioaccessibility in the human body thus giving users access to benefits in the new frontiers of health.

In another embodiment, the particulate bioactive agent includes plant extracts (PBe) including a portion being hydrophobic and already containing natural surface active agents. Some exemplary plant extracts include green tea (Camellia sinensis) or extract having 20% Catechins by weight such as epigallocatechin or epigallocatechin-3-gallate. Another bioactive agent is a combination of a plant extract of turmeric and a plant extract of black pepper in any ratio or combination to form a uniform dry mixture. Specifically, the turmeric extract includes at least 30% of Curcumin (Curcuma longa) by weight and the black pepper extract includes at least 80% Piperine (Piper nigerum) by weight. Yet another bioactive agent includes a plant extract of natto comprising at least 1% of Menaquinone-7 (a fermented Soy product, Glycine max) by weight.

Extracts will never be 100% pure but for Quercetin, for example, a 95% Dihydrate, a 98% Dihydrate and a 99.5% Dihydrate are available. However, the bioactive agent disclosed herein is 100% pure because that is what is being targeted. For the bioactive agent extract (PBe) that contains a minimum percent of bioactive, the lipid soluble portion is targeted. There is no way to only selectively improve the targeted bioactive without additionally improving the others.

The second step, as illustrated in FIG. 1, is to admix or combine the bioactive agent with a lipid to form a mixture 10. The lipid is advantageously also a bioactive for purposes of solubilizing the bioactive in the lipid to improve dispersion of the lipid solubilized bioactive agent in water. The lipid includes fatty acids and glyceride(s) (for example, mono-, di-, tri- or mixtures thereof) of the fatty acids. Exemplary lipids used herein include caproic acid, capric acid (C10), caprylic acid, caproic triglyceride, capric triglyceride, caprylic triglyceride (C8), and mixtures thereof. Mixtures thereof described above are not dependent upon a specific ratio as long as the type of lipids identified herein are used.

The fatty acid preferably includes less than twelve carbons in each fatty acid chain and is free of any carbon-carbon double bonds. Preferably, the fatty acid are saturated fatty acids whereby the fatty acid chain includes all single bonds to advantageously ensure a low rancidity potential of the mixture. Such a configuration advantageously provides an enhanced ability to disperse the lipid in water. Specifically, the fatty acid chain advantageously enhances the characteristics of the bioactive agent by solubilizing the bioactive agent with a lipid to provide beneficial characteristics for a stable formulation and second path metabolism of the bioactive agent with a lipid, which can be converted by cells for cellular energy. Additionally, the fatty acid chain advantageously provides other additional nutritive benefits. Finally, such a configuration advantageously reduces agglomeration.

The lipid can solubilize the bioactive agent or a portion thereof. Specifically, the hydrophobic compound in the bioactive agent is saturated with the lipid such that the lipid completely wets the bioactive to form a slurry.

Additional properties of the lipid preferably include a peroxide value of zero meq/kg, an anisidine value of zero, a moisture content less than 0.05% via weight, and a viscosity range between 11-18 cSt at approximately 40° C. and is liquid at room temperature. These properties advantageously enhance the ability to process the bioactive in a normal commercial industrial setting such as a pharmaceutical, food, beverage, nutraceutical, or agricultural processing facility without the need for additional environmental controls for specific process temperatures and for storage thereafter. Additionally, such properties are advantageously superior for shelf life of finished products that use the invention disclosed herein whether as standalone products or as supplements to other products. Other advantages include reducing spoilage and extending the time to use from manufacturing.

The bioactive agent is combined with the lipid at a ratio of

$$( \frac{bioactive agent}{lipid} < 1 )$$

by weight and preferably at a ratio between 1:1-1:3 and more preferably at a ratio between 1:2-1:3 by weight. This ratio advantageously provides for complete wetting of the bioactive agent. If these ratios are exceeded, the suspension will agglomerate and present various levels of concentration over time. Additionally, enhancing the oil ratio can change palatability and other desired characteristics or possibly alter the intake of dietary lipids beyond recommended daily dietary intake when consuming multiple formulations.

Some plant extract bioactive agents include a lipid composition. For example, the bioactive agent, menaquinone, includes a percentage of lipid that should be measured prior to mixing. The lipid content of the menaquinone is considered when the menaquinone and the lipid are mixed together at a ratio between 2:1 to 1:1 by weight (bioactive:lipid). Measuring the lipid amount and accordingly adjusting the lipid ratio by the content contained in the extract advantageously ensures that the dispersion characteristics are similarly maintained as described above to allow use of a wide variety of extracts, thereby providing an unforeseen benefit. Similarly, a ratio of the mixture based upon the amount of the primary bioactive contained in Catechins, for example, assumes the extract additionally contains other lipophilic compounds, in such case a larger ratio of lipid is used.

As described above, the mixture of the bioactive agent and lipid is mixed until the bioactive agent is fully saturated by the lipid. The mixture is mixed at room temperature but enhanced and accelerated mixing, without any noticeable loss of the properties of the bioactive agent and the lipid, preferably occurs at 65° C. Increased temperatures can further accelerate the mixing process. However, special care should be taken to avoid exceeding volatilization points of the bioactive agent and lipid. The mixture can also be added via a powder incorporation method with an in-line process as understood by one skilled in the art whereby a rotor stator mixer is used that incorporates and disperses the powder. Alternately, the mixture is simply left to self-homogenize with minimal stirring over time.

The lipid advantageously allows for solubility of the bioactive agent, as well as enhances dispersion of the lipid solubilized bioactive agent. The addition of a surface active agent as described below advantageously provides a stable nano or micro-emulsion. The lipid also advantageously acts as a normalizing agent of the bioactive agent within a suspension providing additional stability. Finally, the lipid advantageously provides higher membrane permeability, transport to target cells, and storage and circulation in extracellular fluids. This advantageously improves both transport and secondary path metabolism that are currently lacking among hydrophobic substances used in biochemical applications such as human health, animal health and agriculture. The lipid also provides wide implications to restoration of ecological systems.

FIG. 1 illustrates the third step of adding purified water to the mixture described above to form a suspension 15. Preferably, the added purified water reduces the suspension to a viscosity of 50,000 cP or lower. Specifically, a sufficient amount of water mixes with the bioactive agent and the lipid to form the suspension of the lipid-saturated bioactive agent having solid particles that has a lower viscosity than the initial bioactive-lipid mixture. The function of the water is to reduce agglomerations of the bioactive agent so that the bioactive agent becomes dispersible in water or diffused. The suspension is mixed continuously until the desired reduction in viscosity is achieved and the bioactive agent is fully wetted by the lipid. In addition to wetting, this step additionally removes agglomerations of larger particles.

The reduction in viscosity is temporary to allow for high shear mixing or other comparable method of particle size reduction such as cavitation or high pressure homogenization as understood by one skilled in the art. Also, the step of adding water is performed after any heated mixture has cooled to the point where it can be used effectively in this next process segment. This advantageously ensures that the temperature is reduced to the temperature of the water to avoid lipid phase change, most commonly performed between 20° C.-30° C. (room temperature). In an alternate embodiment, the mixture can simply be mixed without the addition of water to increase solubility and reduce viscosity to achieve similar benefits as described above. As described above, water is added to reduce the viscosity and allow for wetting while reducing particle size. By adding the water, the oil needed for wetting/solubilizing the lipid fully is minimized. At the same time, the surface area of the compound advantageously expands with a minimal amount of lipid added.

The fourth step 20, as illustrated in FIG. 1, is to reduce a particle size of the particulate bioactive agent in the suspension described above to 1 μm or less. The particle size of the bioactive agent is reduced in this step by using, for example, a rotor stator mixer with a specialized head (stator). Preferably, the particle size reduction of the suspension is conducted at room temperature or at a similar temperature of the suspension. This is because alterations to the temperature will disrupt the particle size, distribution, and dispersion of the compounds in the suspension. Additionally, this process of reducing the particle size is performed at a speed less than 3300 rpm, preferably between 1500-3300 rpm, to avoid early dispersion of the suspension prior to the next step.

In one embodiment, the suspension may be left to separate and the separated water is substantially removed to extract other contaminants that may be present in the aqueous phase of the suspension. These other contaminants are advantageously removed to avoid interference with the stability of the final product. While the water is removed, the suspension is still able to be dispersed and added to another equal portion of water to continue with the process. In an alternate embodiment, the water is not separated from the suspension.

In another embodiment, spooning or another removal process understood by one skilled in the art is used to remove any foaming from the surface of the mixture. This removal process advantageously reduces instability and reduces the additional constituents contained within an extract such as polyunsaturated fatty acids and/or additional surface-active agents that may already be unnecessarily present. Additionally, this removal process advantageously contributes to a stable mixture and helps form desired end products in combination with the methods disclosed herein.

The fifth step 25, as illustrated in FIG. 1, is to mix the suspension of the bioactive agent having a particle size of 1 μm or less as described above with a surface active agent to form an emulsion. In the emulsion, the bioactive agent has a particle size of 1.0 μm or less. Both nano and micro-emulsions are defined as complexes which have a mean peak value of the compounds measured by dynamic light scattering of less than one-thousand-five-hundred nanometers (hydrodynamic radii <1500 nm) produced by high-energy or low-energy methods with nano being below 500 nm as a highest range of mean particle distribution in the solution.

The surface active agent advantageously comprises at least one of an inositol, a short chain fructooligosaccharides (sc-FOS) and mixtures thereof. In another embodiment, the surface active agent advantageously consists of at least one of an inositol, a short chain fructooligosaccharides (sc-FOS) and mixtures thereof. Mixtures thereof described above are not dependent upon a specific ratio as long as the type of surface active agent identified herein are used.

Inositols include a modified glucose structure (carbocyclic sugar) whereas the sc-FOS include fructose chains. The sc-FOS preferably includes 1-Kestose, Nystose, or Fructofuranosylnystose. The surface active agent is added to the suspension in a ratio between 10% and 180% and preferably between 10% and 50% by weight of the lipid

$$( \frac{surface active agent}{lipid} ) .$$

This specific range advantageously demonstrates the best improvement to process time and stability. Going above this range can lead to inconsistency, separation and irreversible gelation with high enough energy. The surface active agent preferably is in powder form. Short-chain fructooligosaccharides advantageously include a group of linear fructose oligomers with a degree of polymerization ranging between n=1 to n=5 (also known as oligosaccharides).

The surface active agent defined herein advantageously provides increased stability because of the affinity of the surface active agent to be attracted to both hydrophobic and non-hydrophobic compounds. This attraction allows for enhanced stability in both oil and water and water in oil suspensions. Moreover, since long-chain fructooligosaccharides are more readily available and their enhancements to formulations are known and accepted, the advantage of using the short-chain fructooligosaccharides for the purposes disclosed herein are not seriously considered by the industry until now.

Specifically, the functional uses of long-chain fructooligosaccharides provide for rapid formulation. The benefits of the diverse structures thereof vary widely based on the manufacturer, as well as the plant the long chain fructooligosaccharides were extracted from. Thus, consistent use creates a problem in the industry for short shelf life products, additional microbial growth from separation, and loss of functional properties altogether.

Inositols are synthesized by the body de novo from glucose and are well known for advantageously enhancing biological function and homeostasis of insulin production and enhancing self-homogenization. Inositols have been studied in detail for their role in beneficial cellular health. Specifically, inositols are important contributing factors in a number of biological processes critical to human, animal and plant health. Short-chain fructooligosaccharides are beginning to be studied primarily in the functional benefit in the body in advantageously rebalancing important microorganisms in the GIT and is defined as a prebiotic.

In this fifth step 25, the surface active agent in powder form is added to the solubilized suspension described above. In a preferred embodiment, the surface active agent in powder form is premixed with purified water using a large enough portion of water so that the surface active agent fully dissolves. The water mixture with the dissolved surface active agent is subsequently added to the solubilized suspension. Alternatively, the surface active agent may be mixed at any time in advance with water. This mixture is then measured in the same proportions of the surface active agent required for the lipid as disclosed herein. Nevertheless, the surface active agent and the water premix should not be stored for an extended period of time unless stored under appropriate conditions to prevent microbial activity. This fifth step 25 can be performed at room temperature or the same temperature as the solubilized suspension. However, this step 25 preferably should not be performed at a temperature exceeding 160° C. or the properties of the surface active agent will be compromised.

If water is extracted in the fourth step 20 when the particle size of the solubilized bioactive agent is reduced, preferably any water should be replaced and added before the fifth step 25 of adding the surface active agent powder to the solubilized suspension takes place. When adding the surface active agent powder to the solubilized suspension 25, the surface active agent powder is preferably added slowly to advantageously prevent agglomeration upon solubilizing the surface active agent powder in water. The agglomeration can be avoided significantly by using common powder dispersion process equipment well known in the food and pharmaceutical industry and performing the premixing with water prior to incorporation.

Specifically, this fifth step 25 should preferably be performed with a high shear mixer at a speed dependent on the viscosity of the emulsion. Specifically, the high shear mixer should be used at a speed less than 2500 rpm for emulsions above a viscosity of 500 cps. The high shear mixer should be used at a speed less than 5000 rpm for emulsions below a viscosity of 500 cps. These measures are taken to advantageously prevent the occurrence of gelation which can be irreversible.

Unlike traditional surface-active agents and traditional excipients, the surface active agent disclosed herein advantageously serves additional roles for bioactivity as previously defined. In addition, the surface active agent enhances nano and micro-emulsions form in shearing, separation, stability and reducing separation while functioning simultaneously with the enhancement of bioavailability and bioactivity herein. Moreover, the surface active agent advantageously stabilizes the emulsion so that it can be used in the final formulation.

Finally, FIG. 1 illustrates the last step 30 of finalizing the emulsion through additional processing or use it in its current form for any application. After the fifth step 25, the basic emulsion or suspension is ready. The emulsion or suspension now has enhanced benefits as disclosed herein. Moreover, additional processes can be performed to further enhance form and function.

These methods are more successful than those commonly performed in the industry because of the preparation method described above. Specifically, in addition to the improved stability, the shearing and separation leads to greater homogeneity of the emulsion. As the emulsion is more uniform, the other steps are easier to complete and in some cases (like letting it naturally assemble) the already embodied energy in the emulsion leads to continued assembly. During the continued assembly, the saturated energy (energy sufficiency state) leads to particle groupings of compounds containing the lipid solubilized bioactive agent with the surfactant. The emulsion can be further finalized into a soluble nano, micro-emulsion or suspension by at least one of the following methods.

The first method to finalize the emulsion 30 includes setting, self-assembly or self-homogenizing. This method allows the emulsion to continue to self-assemble and remix, if necessary, until the combination of ingredients in the emulsion forms into a stable liquid matrix. A lower energy sonicating bath may also be used as a mixing method for facilitating self-assembly while setting. Such a method will further enhance the homogeneity and the stability of the mixture over time. This method will also ensure that the saturated energy (energy sufficiency state) is maintained during self-assembly or self-homogenizing.

The second method to finalize the emulsion 30 includes dispersion. In this method, the emulsion is dispersed into another mixture, which can be principally aqueous, using a commonly used dispersion technique such as high pressure homogenization, high energy sonication or another comparable method as understood by one skilled in the art. Using this method, highly desired nano formulations can advantageously be more easily prepared (hydrodynamic radii <200 nm) while, unlike current methods understood by one skilled in the art, process time and energy is drastically reduced. Additionally, a current problem understood in the industry of excessive energy is avoided. Excessive energy can create irreversible forces of gelation or reduce the stability of mixture for long periods of time. As understood by one skilled in the art, the stability post processing in current methods is primarily energetically driven and such energy is known to dissipate over an extended time period.

The third method to finalize the emulsion 30 includes water removal. In this method, moisture content is reduced to 2% to 5% by weight and not to exceed 10% to avoid stability and shelf life issues. Such removal can be accomplished via a commonly used method such as spray drying, freeze drying, or evaporation and milling.

Once the emulsion is finalized 30, the particle size of the lipid solubilized bioactive agent combined with the surface active agent in the emulsion will have a hydrodynamic radii as measured herein approximately 10-1500 nm. This ensures that the bioactive agent, as a constituent, combined with a lipid and surface active agent, is less than 1 micron in particle size with the agglomerated assembly being within the range defined herein.

Preferably, for long term stability, the emulsion will become more stable and a supramolecular assembly (self-assembly by molecular affinity as described herein) will take place when the particle size of the bioactive agent is under 250 nm. Additionally, the particle size of the emulsion can be advantageously adjusted to vary body absorption by adjusting the mixing time and the amount of the surface active agent, whereby larger agglomerations within the range defined herein will have longer retention times and extended release time of the bioactive agent within the GIT.

The emulsion can advantageously act as a stable starting ingredient for creating other products. Additionally, the methods disclosed herein can similarly be followed to advantageously create the other products by starting with a hydrophobic bioactive agent or extract containing such hydrophobic bioactive agent using the methods disclosed herein. Moreover, such complexing can be advantageously used to further enhance products using the methodology disclosed herein. The complexing can be further enhanced by adding hydrophobic ingredients.

An exemplary method according to the invention described herein is as follows. A bioactive agent is a flavonoid having a solubility in water of 0.00215 g/L at 25° C. to 0.665 g/L at 140° C. This example is a method of preparing one liter of this flavonoid, which is 99.5% confirmed by High Pressure Liquid Chromatography (HPLC). The flavonoid, if not ordered to this specification, is milled to pass through a screen of 70 mesh or less. The amount unable to be pass through the 70 mesh is either set aside or discarded.

Next, 100 grams of the screened flavonoid is added to 180 ml (or 156 grams) of a capric (C10) and caprylic (C8) triglyceride lipid liquid concentrate to form a mixture. The powdered flavonoid is then stirred in the liquid lipid until fully wetted or solubilized by the liquid lipid. Afterwards, approximately 400 ml of water is added to the mixture to form a suspension. Subsequently, the suspension is processed with a high shear mixer at 1500-3300 rpm for five minutes.

In a separate container, 420 ml of additional water (this amount of water can be adjusted so long as an emulsion is able to be formed) is combined with a mixture of 25 grams of inositol and 33 grams of sc-FOS to form an emulsion. This emulsion is processed with a high shear mixer or another high shear method to ensure the inositol and/or the FOS are/is completely dissolved using the same manner as described above. Next, the additional water with the dissolved inositol and/or sc-FOS from the separate container is added to the Quercetin/Lipid/Water emulsion and admixed in the same manner as the first water was added.

This process should yield approximately one liter of the ingredient emulsion. As a result of this method, when this ingredient emulsion is added to an aqueous substance, such as a glass of water or a beverage primarily comprising water, the Quercetin is easily dispersed in water and more importantly, better absorbed by the body. Additionally, the preparation of this mixture is advantageous for other uses or additional processing as disclosed herein.

Exemplary hydrophobic ingredients, such as the example of the Quercetin complex, as described above, can be used in cremes, liquids, salves and lotions. Functional beverage powders can also be enhanced using the method disclosed herein. Further, the finalized emulsion can be used as a finished product such as medical food, dietary supplements, mix-ins for beverages and dressings and as food alternatives. The finalized emulsion can also be used, either alone or in combination with the finished product described above, as an inclusion complex for enhancing, for example, several types of current consumer products like those aforementioned, as well as dietary complexes, food and beverage additives, cosmetic ingredients, and in new industry applications, such as agriculture and fisheries.

As commonly understood by one skilled in the art, nano emulsions are excellent carriers for lipophilic bioactive compounds with enhanced properties compared to conventional emulsions. Typically, nano emulsions are defined as oil-in-water (o/w), water-in-oil (w/o) or phase inversions of combinations of both (o/w/to w/o or w/o to o/w) with a very small particle size (r<200 nm). Specifically, when the first compound is dispersed into the second compound, for example an oil-in-water dispersion, the compound dispersed (in the smaller percentage of the solution) is oil dispersed into water. Therefore, one skilled in the art may classify this method as an oil-in water emulsion method.

Alternatively, depending on the amount of water added in the step of adding water (to reduce viscosity), by adding a smaller amount of water to the lipid solubilized bioactive compound, a phase inversion occurs. In this phase inversion, the water is first added into a higher percentage of oil-water-in-oil. Subsequently, when the additional water and surface active agent are added, it becomes oil-in-water.

The small particle dimensions advantageously confer unique properties such as improved physical stability, high optical clarity, and enhanced bioavailability. As a result of the small particle size, nano emulsions have a large surface area and can therefore interact strongly with biological components in the GIT while significantly reducing the waste or toxicity that comes from hydrophobic drugs with poor absorption. Accordingly, much smaller quantities are effective and less of the compound is wasted or unused in the GIT.

The method and formulations described herein for preparation of mass quantities of nano and micro-emulsions and suspensions advantageously applies a size reduction method whereby the limitations of the traditional nano emulsion process can be overcome. Such limitations include the difficulties of instability in large batches, the inability to produce large volumes of materials cost efficiently, and the relatively low loading rates of bio actives whereby a minimum effective dose for disease treatment is difficult to achieve.

The method and formulation described herein advantageously advances many methods available for study in the research community and develops a new version of “food as medicine.” The “food as medicine” includes the bioactivity necessary that can advantageously alter the trajectory of the diseased state that afflicts much of the human population today. The “food as medicine” is also advantageously different from synthetic ingredients which are the common primary additions for preparation enhancement and stability in nano formulations, and used as stabilizing agents. The “food as medicine” also advantageously uses only ingredients that also function as co-nutrients to align with consumers desires for a clean label and provides eco-conscious solutions for health and wellbeing.

Formation of food-grade nano and micro-emulsions can be divided into high-energy or low-energy methods. High-energy approaches use mechanical forces to intermingle oil and aqueous phases and produce smaller particles. Exemplary high-energy methods include high-pressure homogenization, micro-fluidization, and sonication. Low-energy methods use changes in the composition or environment of a surfactant-oil-water system to spontaneously form small particles. Exemplary low-energy methods include spontaneous emulsification or some phase-inversion methods. Low-energy methods are able to produce nano emulsions with simple equipment and avoid the temperature increase that is caused when using many high-energy approaches, with similar particle size and stability characteristics.

In view of the above, it is important to select the optimal fabrication method to obtain nano and micro-emulsions with optimal properties. Nano emulsions can be formulated with triacylglycerol oils, flavor oils, essential oils, mineral oils, or waxes, for example. Oils with different fatty acid compositions advantageously provide significantly different lipid digestion rates, complicating bioactivity, as well as leading to mixed micelle phases with appreciably different solubilization capacities for a hydrophobic bioactive. Long chain triglycerides are advantageously digested more slowly than medium or short chain triglycerides. Moreover, surfactants and emulsifiers used to formulate nano emulsions also advantageously impact the solubilization capacity of bioactive compounds. Size reduction and removal of agglomerations in nutritive substances can advantageously enhance the body's access to such substance.

Of particular interest in biology and physiology is the preparation of nano substances that can reduce human waste and maximize the use of the nutrient for the organism. Specifically, a diminished liver function across the world among the human population from dietary factors, medications, and other environmental influences is problematic for human health. It is estimated that in the US alone 100,000,000 Americans have NAFLD (non-alcoholic fatty liver) and annually 700,000 Americans have gallbladder removal surgery.

As the liver is the essential organ for metabolism and the gallbladder is additionally important for biosynthesis, the method disclosed herein provides a solution to the health and dietary challenges. In particular, the method disclosed herein advantageously assimilates nutrients beneficial for health that pre-assimilates compounds and delivers them in such a way where they can reach the lymphatic system and the circulatory system without need for additional biosynthesis from the liver or gallbladder. Specifically, these benefits are advantageously realized by solubilizing the bioactive agent in a specific lipid and reducing the size to where the bioactive agent can move rapidly across membranes. Additionally, the specific surface active agent in sc-FOS advantageously helps with delivery in the GIT so that the lipid solubilized bioactive agent can be pulled into the lymphatic system.

Nano and micro-preparations commonly used in the industry have shown promise but have been held back as a primary system in industry for ingredient preparation for the following reasons: the cost of preparation, using primarily synthetic components for assembly, poor stability, and poor loading capabilities of a primary bioactive making them difficult to use for nutritive compounds that have low toxicity. These primary bio actives exist in large quantities in nature but lack the ability as an extract to be easily assimilated or absorbed by the body. Additionally, limiting the use of nano and micro-emulsions contributes to the cost and complexity of the equipment and process necessary to integrate such method as a primary method of nutrient enhancement of hydrophobic substances.

Finally, a primary obstacle for advancement of such preparation methods is the poor loading rates of such compounds, which amplify the cost versus reward conundrum aforementioned. In other words, the preparation method disclosed herein advantageously provides a new means that overcomes the primary obstacles of preparations commonly understood in industry. Specifically, the preparation method disclosed herein advantageously provides value-added ingredients and products for health that align with the capabilities of current consumers, government, or the health system to pay for such value addition.

The advantageous preparation method disclosed herein provides a cost effective solution formulated with natural compounds that are abundantly and consistently provided by nature. Additionally, new mechanical systems and processes are advantageously applied via the method disclosed herein to scale the creation of this advantageous emulsion and its benefits. The method disclosed herein departs from the strategy commonly understood in the prior art of the benefits and enhances the ability of such solutions to become a primary method of low toxicity treatment of specific diseases using particle sizes to enhance efficacy and show revolutionary results on different disease with natural products. Specifically, on the formulation side, the focus of the industry has been to emulate the same method or process repeatedly with small changes. On the production side, the focus of the industry has been on sonication, high energy methods of preparation, and other methods which are challenging to scale.

Moreover, these natural compounds are well known to have positive effects of being anti-cancer, anti-viral, cardioprotective, neuroprotective, and being theorized as complete solutions to current pandemics if bioavailibilty and second path metabolism is addressed.

The preparation method disclosed herein advantageously provides an advanced methodology for drying preparations by improving the structure of the formulation to enhance the agglomeration consistency from drying. While drying methods are well known, this invention as a preparatory method and formulary enhancement favorably alters the characteristics of the compound to be dried so agglomeration during drying leads to an enhancement of uniform particles that can have additional use in enhancing the efficacy of dried powders for new applications, such as medical inhalation or dry powdered dispersion in water at a later date.

Such improvements enhance the industrial use of the compound, as well as provide an extended shelf life and improved physical chemical characteristics. The method disclosed herein reduces the potential for spoilage and also enhancing the ability for use with non-wetted products. Additionally, drying removes water weight for enhancing logistics outcomes. Such consistency is made possible by the pre-assembly method disclosed herein by first solubilizing the bioactive agent in the lipid followed by surrounding that lipid solubilized bioactive agent with a surface active agent. Drying may require additional drying agents which can be suspended in the polar solvent (such as water). Contrary to current industry applications, by using wholly bioactive compounds in the suspension, the focus on the finished water soluble products goes beyond flowability and blend-ability of powders. The current industry method to add starches as drying and dispersion aids is not preferred because this method adds readily metabolized sugars without providing health benefits. In fact, such a method creates negative health outcomes which can interfere with bioactivity for specific uses.

The preparation method disclosed herein advantageously advances the aglycone bioavailability and bioactivity to relieve the limitations that limit the ability to be used in nano and micro-complexes because of extreme hydrophobicity. Additionally, the added bioavailability and bioactivity from the functional enhancements herein overcome the limitations of poor GIT absorption of well-known transformative natural health compounds that can now be transferred into both lymphatic and circulatory systems, absorbed into the cell, and the ability to achieve a loading rate that reaches a minimum effective dose to be active. On the other hand, the industry approach of enhancing bioavailability alone by a chemical preparation method of adding bonded moieties can interfere with bioactivity for specific uses.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the precise embodiments disclosed. Any of the embodiments and/or elements disclosed herein may be combined with one another to form various additional embodiments not specifically disclosed, as long as they do not contradict each other. Accordingly, additional embodiments are possible and are intended to be encompassed within this specification and the scope of the invention. It will be understood by one skilled in the art that this disclosure is not limited in its application to the details of the components or order of the method steps set forth in the above description or illustrated in the drawings. The embodiments herein are capable of other embodiments, and capable of being practiced or carried out in various ways. The specification describes specific examples to accomplish a more general goal that may be accomplished in another way.

It will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Terms of degree, such as “substantially” or “approximately” are understood by those of ordinary skill to refer to reasonable ranges outside of the given value, for example, general tolerances associated with manufacturing, assembly, and use of the described embodiments.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A method of preparing an emulsion of a bioactive agent having enhanced bioavailability, the method comprising:
admixing a particulate bioactive agent with a lipid to form a mixture;
adding water to the mixture to form a suspension of the bioactive agent and the lipid;
reducing a particle size of the bioactive agent in the suspension to 1.0 μm or less; and
adding a surface active agent to the resulting suspension of the bioactive agent to create the emulsion containing the bioactive agent having a particle size of 1.0 μm or less.

2. The method of claim 1, wherein the lipid includes at least one selected from the group consisting of fatty acids and glycerides of fatty acids.

3. The method of claim 2, wherein the lipid includes one of a caproic acid, capric acid, caprylic acid, caproic triglycerides, capric triglycerides, caprylic triglycerides, and mixtures thereof.

4. The method of claim 2, wherein
the fatty acids include 12 or fewer carbon atoms;
the glycerides include one of monoglycerides, diglycerides, and mixtures thereof; and
the fatty acids include saturated fatty acids.

5.-6. (canceled)

7. The method of claim 1, wherein
the particulate bioactive agent mixed with the lipid has a particle size of 210 μm or less; and
the bioactive agent and lipid are combined in a ratio of about 1:1 (bioactive:lipid) by weight to about 1:3 (bioactive:lipid) by weight.

8. (canceled)

9. The method of claim 1, wherein the water is added to the mixture of the particulate bioactive agent and the lipid to adjust the viscosity of the suspension to 50,000 cP or less.

10. The method of claim 1, wherein the particulate bioactive agent comprises a concentration of at least 20% by weight of a hydrophobic compound with a solubility less than 5 mg per ml in water.

11. The method of claim 10, wherein the hydrophobic compound includes quercetin.

12. The method of claim 10, wherein the hydrophobic compound includes a anthocyanin.

13. The method of claim 10, wherein the hydrophobic compound includes a vitamin group D.

14. The method of claim 11, wherein the quercetin comprises a quercetin aglycone.

15. The method of claim 1, wherein the particulate bioactive agent includes Catechins comprising one of epigallocatechin and epigallocatechin-3-gallate.

16. The method of claim 1, wherein the surface active agent is added to the suspension in an amount of 10% to 180% by weight based on the weight of the lipid.

17. The method of claim 1, wherein the surface active agent is selected from the group consisting of inositol, short chain fructooligosaccharides, and mixtures thereof.

18. The method of claim 1, wherein the surface active agent is a composition comprising one of inositol, short chain fructooligosaccharides, and mixtures thereof.

19. The method of claim 18, wherein the short chain fructooligosaccharides comprises 1-Kestose, Nystose, Fructofuranosylnystose or mixtures thereof.

20. The method of claim 1, further comprising one of dispersing the emulsion in water to form an emulsified mixture and drying the emulsion by removing the water to form a powder using freeze drying, or evaporation and milling.

21. The method of claim 1, further comprising:
mixing the suspension with the surface active agent using a high shear mixer to prevent gelation based on the following conditions:
the high shear mixer operating at a speed less than 2500 rpm with mixtures above 500 cps; and
the high shear mixer operating at a speed less than 5000 rpm with mixtures below 500 cps.

22. (canceled)

23. A method of preparing an emulsion having enhanced bioavailability, the method comprising:
preparing a particulate bioactive agent comprising a particulate plant extract of green tea including at least 20% catechins by weight and having a particle size of 210 μm or less;
mixing the particulate bioactive agent with a lipid selected from the group comprising one of caproic acid, capric acid, caprylic acid, caproic triglycerides, capric triglycerides, caprylic triglycerides, and mixtures thereof to form a mixture at a ratio between 1:2 to 1:3 by weight (bioactive:lipid);
saturating the particulate mixture in the lipid;
adding water to the mixture to form a suspension of the lipid saturated bioactive agent;
reducing a particle size of the particulate bioactive agent in the suspension to 1.0 μm or less; and
adding a surface active agent to the resulting suspension to create an emulsion including the bioactive agent having a particle size of 1.0 μm or less, where the surface active agent is selected from the group comprising one of inositol, short chain fructooligosaccharides, and mixtures thereof in a ratio between 0.1 to 0.5 by weight of the lipid; wherein
the emulsion comprises the particulate plant extract having a portion of the particulate bioactive agent being hydrophobic.

24. A method of preparing an emulsion having enhanced bioavailability, the method comprising:
preparing a bioactive agent including a first particulate plant extract of turmeric comprising at least 30% of curcumin by weight and having a particle size of 210 μm or less, and including a second particulate plant extract of black pepper comprising at least 80% piperine by weight and having a particle size of 210 μm or less;
mixing the first and second particulate plant extracts to form a uniform dry mixture;
mixing the dry mixture with a lipid selected from the group comprising one of caproic acid, capric acid, caprylic acid, caproic triglycerides, capric triglycerides, caprylic triglycerides, and mixtures thereof;
saturating the particulate mixture in the lipid;
adding water to the mixture to form a suspension of the lipid saturated bioactive agent;
reducing a particle size of the particulate bioactive agent in the suspension to 1.0 μm or less; and
adding a surface active agent to the resulting suspension to create an emulsion including the bioactive agent having a particle size of 1.0 μm or less, where the surface active agent is selected from the group comprising one of inositol, short chain fructooligosaccharides, and mixtures thereof; wherein
the emulsion comprises the plant extract having a portion of the bioactive agent being hydrophobic.

25.-36. (canceled)
